SEQUENCE LISTING (2 sequences)

SEQ ID NO: 1 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 2 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

(19) United States
(12) Patent Application Publication
(10) Pub. No.: US 2013/0197106 A1
(43) Pub. Date: Aug. 1, 2013

(54) METHODS OF IDENTIFYING A CANDIDATE COMPOUND

(71) Applicants: Valeria Fantin, La Jolla, CA (US); Shin-San Michael Su, Newton, MA (US)

(72) Inventors: Valeria Fantin, La Jolla, CA (US); Shin-San Michael Su, Newton, MA (US)

(73) Assignee: AGIOS PHARMACEUTICALS, INC, Cambridge, MA (US)

(21) Appl. No.: 13/638,881

(22) PCT Filed: Mar. 31, 2011
(86) PCT No.: PCT/US11/30692

§ 371 Date: Jan. 4, 2013

Related U.S. Application Data
(60) Provisional application No. 61/320,255, filed on Apr. 1, 2010.
(60) Provisional application No. 61/331,322, filed on May 4, 2010.

(51) Int. Cl. C12Q 1/32

(52) U.S. Cl. CPC C12Q 1/32
USPC 514/789; 435/32; 435/26; 506/10

(57) ABSTRACT

The invention relates to methods identifying compounds for the treatment of cell proliferation-related disorders, e.g., proliferative disorders such as cancer.

### CLAIM OF PRIORITY

This application claims priority from U.S. Ser. No. 61/320,255, filed Apr. 1, 2010 and U.S. Ser. No. 61/331,322, filed May 4, 2010, each of which is incorporated herein by reference in its entirety.

### FIELD OF THE INVENTION

The invention relates to methods of identifying compounds for the treatment of cell proliferation-related disorders, e.g., proliferative disorders such as cancer.

### BACKGROUND

Isocitrate dehydrogenase, also known as IDH, is an enzyme that catalyzes the oxidative decarboxylation of isocitrate to 2-oxoglutarate (i.e., α-ketoglutarate or α-KG). These enzymes belong to two distinct subclasses, one of which utilizes NAD(+) as the electron acceptor, and the other which utilizes NADP(+). Five isocitrate dehydrogenases have been reported, two of which (IDH1 and IDH2) are NADP(+)-dependent. IDH1 is expressed in the cytoplasm and peroxisomes, and IDH2 is expressed in the mitochondria.

Malignant gliomas, including primary and secondary glioblastomas, are among the most lethal with median survival of one year, and unfortunately also the most prevalent of brain tumors. Unbiased genomic analysis of >20K genes for 22 glioma genomes found recurrent mutations of IDH1 on chromosome 2q33. Recurrent IDH mutations have been identified in up to 70% of grade II-IV gliomas, in about 10% of AML cases, and in several other cancer types at lower-frequencies. Common somatic mutations of IDH1/2 found thus far have been heterozygous point substitutions at codon 132 (or R172/R140 in IDH2), which result in loss-of-function for metabolizing isocitrate. The IDH1^{R132H} mutantion confers a gain-of-function to produce the oncometabolite 2-hydroxyglutarate (2HG), and in effect defining IDH1 and IDH2 as oncogenes.

All IDH1 132 and IDH2 172 mutations identified to date share a common neomorphic activity resulting in production of D-2HG. The rare neurometabolic disorder known as 2-hydroxyglutaric acidura (2HGA) is characterized by elevated levels of D-2HG or L-2HG due to germline mutations in either D-2HG or L-2HG dehydrogenases, and affected individuals have been shown to be predisposed to malignant brain tumors. Thus, it is likely that the D-2HG production associated with IDH1 and IDH2 mutations is the common gain-of-function that leads to neoplasia in tumor cells carrying the IDH mutations.

### SUMMARY OF THE INVENTION

The invention is based at least on the discovery of a method for identifying a compound that interferes with proliferation or viability of an IDH (isocitrate dehydrogenase)-mutant cell overexpressing 2HG (2-hydroxyglutarate). The method includes contacting a candidate compound with an IDH-mutant cell that has elevated levels of 2HG, and if proliferation or viability of the IDH-mutant cell that has elevated levels 2HG is decreased as compared to a control cell that does not have elevated levels of 2HG, then the candidate compound is selected as a compound that specifically interferes with proliferation of the IDH-mutant cell.

The IDH-mutant cell can carry a mutation in the IDH1 or the IDH2 gene. For example, the mutant cell can carry an IDH1^{R132X} mutation, an IDH2^{R172X} mutation, or an IDH2^{R140X} mutation, where “X” represents any amino acid residue. For example, the mutant cell can carry an IDH1^{R132H} mutation, an IDH1^{R132C} mutation, an IDH2^{R172K} mutation, an IDH2^{R140Q} mutation, or any mutation shown in Table 1.

The IDH-mutant cell is typically from a cancer cell line, such as a glioma (e.g., a U87 cell line) or a fibrosarcoma (e.g., an HT1080 cell line), or a cell line described in Table 1. For example, the IDH-mutant cell can be from a U87 glioma cell line engineered to express IDH1^{R132X}, (e.g., IDH1^{R132H}) or from a fibrosarcoma HT1080 cell line that carries an IDH1^{R132C} mutation.

The selection assays featured in the invention can include a primary assay and at least one secondary assay. For example, a primary assay can include determining whether a compound can selectively inhibit proliferation or viability of one type of IDH-mutant cell line that has elevated levels of 2HG, and if the compound does selectively inhibit proliferation or viability, then the compound can be tested in a secondary assay against a different type of IDH-mutant cell line that has elevated levels of 2HG. If the compound selectively inhibits proliferation or viability of the second type of cell-line, then the compound can be selected for further study, such as for its suitability as a therapeutic agent to treat a proliferative disorder, such as a cancer.

In another embodiment, the secondary assay is a mechanism-based assay. For example, the secondary assay can include determining whether a compound can specifically cause decreased levels of 2HG from the IDH-mutant cell line, and if the compound does cause decreased levels, then the compound can be selected/identified for further study, such as for its suitability as a therapeutic agent to treat a cancer.

In another embodiment, the primary assay is a mechanism-based assay, and the secondary assay is a phenotype-based assay. For example, the primary assay can include determining whether a compound can specifically cause decreased levels of 2HG from an IDH-mutant cell line, and if the compound does cause decreased production, then the compound can be tested in a secondary assay, which is a phenotype-based assay, against the same or a different type of IDH-mutant cell line that has elevated levels of 2HG. If the compound selectively inhibits proliferation or viability of the cell line, then the compound can be selected for further study, such as for its suitability as a therapeutic agent to treat a cancer. In this embodiment, a candidate compound can act specifically on the mutant IDH enzyme to cause the decreased prodution of 2HG.

The candidate compound can be useful for selectively targeting tumors or treating cancers characterized by IDH mutants that cause overexpression of 2HG. The candidate compounds identified by the selection methods featured in the invention can be further examined for their ability to target a tumor or to treat cancer by, for example, administering the compound to an animal model.

Further, a candidate compound identified by the selection methods described herein can be formulated in a pharmaceutical formulation and administered to a human to treat a cancer, such as a cancer characterized by an IDH mutation, and by cancer cells that overexpress 2HG.

Other features and advantages of the invention will be apparent from the following detailed description, the drawings, and the claims.

### BRIEF DESCRIPTION OF THE DRAWINGS

FIG. 1 is the amino acid sequence of IDH1 as described in GenBank Accession No. NP_{—}005887.2 (Record dated May 10, 2009; GI No. 28178825) (SEQ ID NO:1).

FIG. 2 is the amino acid sequence of IDH2 as presented at GenBank Accession No. NP_{—}002159 (Record dated Apr. 19, 2010; GI No. 28178832) (SEQ ID NO:2).

FIG. 3 is a graph depicting a kinetic analysis of isocitrate consumption of isocitrate by the mutant IDH1^{R132II}, while recycling occurs between NADP and NADPH.

FIG. 4 is a graph depicting a time course of IDH1R132H enzyme activity at various enzyme concentrations.

FIG. 5 is a graph depicting IC50 determination for suramin, a non-specific dehydrogenase inhibitor.

FIGS. 6A and 6B are graphs depicting the consistency of enzyme assays when IDHR132H mutant enzyme was incubated in the presence and absence of suramin.

FIGS. 7A and 7B are graphs depicting the consistency of cell viability assays performed by testing the effect of DMSO control and the general cytotoxic agent staurosporine on mutant engineered U87MG-R132H (FIG. 7B) or parental cells (FIG. 7A).

FIG. 8 is a graph depicting the dose-responsive effect of staurosporine on viability of U87MG^{R132H} mutant cells.

### DETAILED DESCRIPTION

The invention is based at least on the discovery of a method for identifying a compound that interferes, e.g., selectively interferes, with proliferation or viability of an IDH (isocitrate dehydrogenase)-mutant cell overexpressing 2HG (2-hydroxyglutarate). The method includes contacting a candidate compound with an IDH-mutant cell that has elevated levels of 2HG, and if proliferation or viability of the IDH-mutant cell that has elevated levels of 2HG is decreased as compared to a control cell that does not have elevated levels of 2HG, then the canditate compound is selected as a compound that selectively interferes with proliferation or viability of the IDH-mutant cell.

Isocitrate Dehydrogenases

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2, 4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

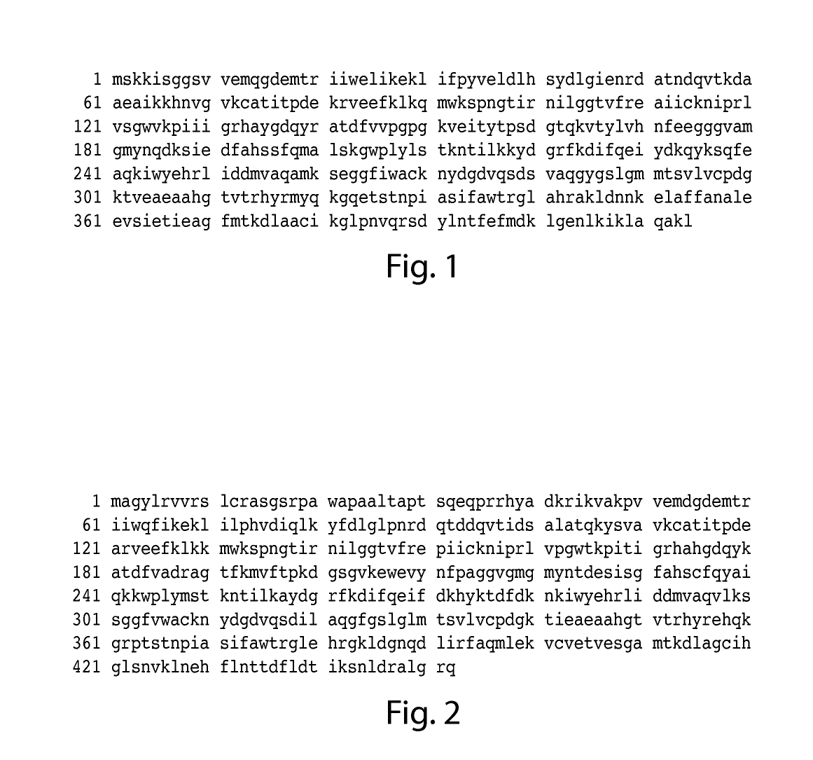

The human IDH1 gene encodes a protein of 414 amino acids. The nucleotide and amino acid sequences for human IDH1 can be found as GenBank entries NM_{—}005896.2 and NP_{—}005887.2, respectively (see FIG. 1). The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMBL/GenBank/DDBJ databases; and Sjoeblom et al., Science 314:268-274(2006).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_{—}002168.2 and NP_{—}002159.2, respectively (see FIG. 2). The nucleotide and amino acid sequence for human IDH2 are also described in, e.g., Huh et al., Submitted (NOV-1992) to the EMBL/GenBank/DDBJ databases; and The MGC Project Team, Genome Res. 14:2121-2127(2004).

Non-mutant, e.g., wild type, IDH1 catalyzes the oxidative decarboxylation of ioscitrate to α-ketoglutarate thereby reducing NADP⁺ to NADPH, e.g., in the forward reaction:

Isocitrate+NADP⁺→α-KG+CO₂+NADPH+H⁺

In some embodiments, the mutant IDH1 and/or IDH2 (e.g., a mutant IDH1 and/or IDH2 having a neoactivity described herein) could lead to an elevated level of 2-hydroxyglutarate, e.g., R-2-hydroxyglutarate, or D-2-hydroxyglutarate or L-2-hydroxyglutarate, in a subject. The mutant IDH1 or IDH2 can have a neoactivity, which is the reduction of αKG to 2HG as follows:

α-KG+NADPH+H⁺→2-hydroxyglutarate, e.g., R-2-hydroxyglutarate+NADP⁺.

The accumulation of 2-hydroxyglutarate, e.g., R-2-hydroxyglutarate in a subject, e.g., in the brain of a subject, can be harmful. For example, in some embodiments, elevated levels of 2-hydroxyglutarate, e.g., R-2-hydroxyglutarate can lead to and/or be predictive of cancer in a subject such as a cancer of the central nervous system, e.g., brain tumor, e.g., glioma, e.g., glioblastoma multiforme (GBM). Accordingly, in some embodiments, a method described herein includes administering to a subject an inhibitor of the neoactivity.

In some embodiments, the neoactivity can be the reduction of pyruvate or malate to the corresponding α-hydroxyl compounds.

In some embodiments, the neoactivity of a mutant IDH1 can arise from a mutant IDH1 having a His, Ser, Cys, Gly, Val, Pro or Leu, or any other mutations described in Yan et al., at residue 132 (e.g., His, Ser, Cys, Gly, Val or Leu; or His, Ser, Cys or Lys). In some embodiments, the neoactivity of a mutant IDH2 can arise from a mutant IDH2 having a Lys, Gly, Met, Trp, Thr, or Ser (e.g., Lys, Gly, Met, Trp, or Ser; or Gly, Met or Lys), or any other mutations described in Yan H et al., at residue 172. Exemplary mutations include the following: R132H, R132C, R132S, R132G, R132L, R132V, and/or any of the mutations described in Table 1.

TABLE 1
IDH mutations for use in screening methods.

| Cancer Type | IDH1 | IDH2 | Tumor Type |
|---|---|---|---|
| brain tumors | R132H | R172K | primary tumor |
| | R132C | R172M | primary tumor |
| | R132S | R172S | primary tumor |
| | R132G | R172G | primary tumor |
| | R132L | R172W | primary tumor |
| | R132V | | primary tumor |
| colon cancer | G97D | G137D | HCT15, DLD colon |
| | | | cancer cell line |
| fibrosarcoma | R132C | | HT1080 fibrosarcoma |
| | | | cell line |
| Acute Myeloid | R132H | R140Q | primary tumor |
| Leukemia (AML) | R132G | R172K | primary tumor |
| | R132C | | primary tumor |
| Prostate cancer | R132H | | primary tumor |
| | R132C | | primary tumor |
| Acute lymphoblastic | R132C | | primary tumor |
| leukemia (ALL) | | | |
| paragangliomas | R132C | | primary tumor |

Screening Methods The invention includes methods for identifying a compound that interferes with, e.g., selectively interferes with, proliferation or viability of an IDH (isocitrate dehydrogenase)-mutant cell overexpressing 2HG (2-hydroxyglutarate). The method includes contacting a candidate compound with an IDH-mutant cell that has elevated levels of 2HG, and if proliferation or viability of the IDH-mutant cell that has elevated levels of 2HG is decreased as compared to a control cell that does not have elevated levels of 2HG, then the candidate compound is selected as a compound that selectively interferes with proliferation of the IDH-mutant cell. The methods described herein can be used applied in a high throughput format to a compound library.

The type of screen described above can be referred to as a “synthetic lethal” screen, which identifies a compound that selectively inhibits proliferation or viability of a mutant cell, but does not affect proliferation or viability of a wildtype cell, or a cell that otherwise does not carry the same mutation.

As used herein, the term “selectively” or “selective” is meant that a compound acts to a greater extent on the proliferation or viability (and/or the ability to produce 2-HG) of a cell carrying an IDH mutation than on a cell that does not carry the mutation. For example, the effect of the compound on an IDH-mutant cell is at least 20%, 50%, 75%, 2-fold, 3-fold, 4-fold, 5-fold, 6-fold, or 10-fold or more greater than its effect on a cell that does not carry the IDH mutation. Thus, in some embodiments, the compound selectively inhibits proliferation or viability of the IDH-mutant cell, and does not inhibit (or inhibits to a much lesser extent) proliferation or viability of a cell that does not carry the IDH mutation. For example, a compound can be selective for a U87 cell engineered to express the IDH1^{R132H} mutation, but not the parental U87 cell.

As used herein, the term “specifically” or “specific” is meant that a compound acts to a greater extent on the activity of a mutant IDH enzyme than on another cellular target, such as another enzyme, e.g., a wildtype IDH enzyme, or an IDH enzyme that does not carry the same mutation. For example, the effect of the compound on a mutant IDH enzyme is at least 20%, 50%, 75%, 2-fold, 3-fold, 4-fold, 5- fold, 6-fold, or 10-fold or more greater than its effect on an IDH enzyme that does not carry the same IDH mutation. Thus, in some embodiments, the compound specifically inhibits the ability of the mutant IDH enzyme to produce 2HG, and does not inhibit (or inhibits to a much lesser extent) the ability of the non-mutant IDH enzyme to produce 2HG. For example, a compound can be specifically for an IDH1 enzyme carrying an IDH1^{R132H} mutation, but not the wildtype IDH1 enzyme.

A compound that inhibits “proliferation or viability” slows or stops the division of cells, or kills the cells.

As used herein, an IDH mutant cell that has “elevated levels” of 2HG produces (e.g., secretes from the cell) 10%, 20% 30%, 50%, 75% or more 2HG then the same cell that does not carry the IDH mutation.

As used herein, the terms “inhibit” or “prevent” include both complete and partial inhibition or prevention. An inhibitor may completely or partially inhibit.

The selection method featured in the invention can utilize any cell line that carries a mutant IDH gene that correlates with elevated levels of 2HG. For example, the cell lines can be cancer cell lines, such as cell lines derived from a solid tumor, such as of the brain, thyroid, colon, prostate, or bone, or from a hematological cancer, such as multiple myeloma, leukemia, lymphoma. Cell lines can originate from tumors and other cancers as shown in Table 1 above.

Screening methods can include a primary screen and at least one secondary assay. For example, a primary assay can include determining whether a compound can selectively interfere with (e g inhibit or reduce) proliferation or viability of one type of IDH-mutant cell line that has elevated levels of 2HG, and if the compound does selectively interfere with (e.g. inhibit or reduce) proliferation or viability, then the compound can be tested in a secondary assay against a different type of IDH-mutant cell line that has elevated levels of 2HG. If the compound selectively inhibits proliferation or viability of this second type of cell-line, then the compound can be selected for further study, such as for its suitability as a therapeutic agent to treat a cancer.

For example, in a primary selection assay, a compound can be tested for its ability to selectively interfere with (e.g. inhibit or reduce) proliferation or viability of U87 cells engineered to express IDH1^{R132H}, and if the compound does selectively interfere with (e.g. inhibit or reduce) proliferation or viability, then the compound can be tested in a secondary assay against HT1080 cells, which express IDH1^{R132C} mutant enzyme. If the compound selectively interferes with (e g inhibit or reduces) proliferation or viability of this second type of cell-line, then the compound is selected for further study, such as in animal cancer models for its suitability as a therapeutic agent to treat a cancer.

In some embodiments, one assay, e.g., the primary assay is a mechanism-based assay, and another assay, e.g., the secondary assay, is a phenotype-based assay. For example, the primary assay, which is a mechanism-based assay, can include determining whether a compound can specifically cause decreased production of 2HG from an IDH-mutant cell line, and if the compound does cause decreased production, then the compound can be tested in a secondary assay, which is a phenotype-based assay, against the same or a different type of IDH-mutant cell line that has elevated levels of 2HG. If the compound selectively interferes with (e g inhibits or reduces) proliferation or viability of the cell line, then the compound can be selected for further study, such as for its suitability as a therapeutic agent to treat a cancer.

For example, the primary assay can be performed by contacting an IDH-mutant cell, such as a U87 cell engineered to express IDH1^{R132H}, with a candidate compound, and determining whether the amount of 2HG expressed by the cell is decreased. Expression of 2HG can be determined, for example, by an enymatic fluorescence-based assay, or by LC-MS/MS (liquid chromatography-mass spectroscopy/mass spectroscopy. If the compound is determined to cause decreased 2HG production, then the compound can be tested for its ability to interfere with (e.g. inhibit or reduce) proliferation or viability of the U87-IDH^{R132H} cells. If the compound is found to selectively interfere with (e g inhibit or reduce) proliferation or viability of the cell line, then the compound can be selected for further study, such as for its suitability as a therapeutic agent to treat a cancer.

The primary and/or secondary assay can include a synthetic lethal screen that utilizes a cell line engineered to express a neoactive IDH mutant that causes elevated levels of 2HG. A compound that selectively causes decreased proliferation or viability of cells the engineered cell line, as compared to cells from a control cell line, e.g., the parent (non-engineered) cell line, will be selected as a candidate compound that may be useful for treatment of a proliferative disorder, such as a cancer.

In one alternative, the secondary assay, or a further confirmatory assay, the identified compound is tested for its ability to inhibit the viability of neurospheres of glioma cells derived from glioblastoma patients whose tumors harbor an IDH mutation, such as the IDH1^{R132H} mutation.

In some embodiments, the mechanism-based assay is the primary assay, and the phenotype-based assay is the secondary assay. In other embodiments, the phenotype-based assay is the primary assay, and the mechanism-based assay is the secondary assay.

In one embodiment, a compound that is identified as selectively causing decreased proliferation or viability of the engineered cell line can be used in a secondary assay to confirm the effect on cell proliferation or viability on other IDH mutant cells, or to determine whether the compound is capable of decreasing 2HG production from the neoactive IDH mutant cells. In another embodiment, primary assay can include determining whether a compound can specifically cause decreased production of 2HG from an IDH-mutant cell line, and if the compound does specifically cause decreased production, then the compound can be tested in a secondary assay against the same or a different type of IDH-mutant cell line that has elevated levels of 2HG. If the compound selectively inhibits proliferation or viability of the cell-line, then the compound can be selected for further study, such as for its suitability as a therapeutic agent to treat a cancer.

Control cell lines used in the selection assays featured in the invention are as similar as possible to the test cell line, i.e., the IDH-mutant cell line that has elevated levels of 2HG. For example, a cell line engineered to express an IDH mutant transcript can be used as a test cell line, and the control cell would typically be the parent non-engineered cell line. In another example, a cell line that carries an endogenous IDH mutation that confers a gain of function phenotype that leads to overexpression of 2HG can be used as a test cell line, and in this case, the control cell line would be the same cell line, engineered such that 2HG is not overexpressed. For example, the control cell line can be engineered to express a nucleic acid (e.g., a microRNA, siRNA or antisense RNA) that inhibits expression of the IDH mutant transcript.

Compounds for Screening

The compounds suitable for use in the selection methods featured in the invention can be artificial or synthetic. The compounds can be obtained from a library, such as commercial library, and the compounds can be synthesized and assembled into a library, such as for use in a high through-put screen.

A compound that selectively inhibits proliferation or viability of an IDH mutant cell may act through a variety of mechanisms. For example, the compound may inhibit 2HG export from the cell, increase oxidative stress in the cell, or interact with 2HG to produce a toxic derivative of 2HG or the candidate compound.

A candidate compound that selectively interfers (inhibits or reduces) proliferation or viability of a cell can be selected for further study if the compound inhibits (e.g., slows or stops) division of, or kills, the IDH-mutant cells that have elevated levels of 2HG, but does not inhibit proliferation or viability (or kill), cells from a control cell line. The control cell line may be a parental cell type that does not carry the IDH mutation, or the control cell line may be the IDH-mutant cell line that has been engineered such that levels of 2HG are not elevated.

For example, a candidate compound suitable for further study inhibits proliferation or viability of cells from a U87 glioma cell line engineered to express IDH1^{R132H} but has no effect on the proliferation or viability of cells from the parent U87 cell line. In another example, a candidate compound suitable for further study inhibits proliferation or viability of cells from a fibrosarcoma HT1080 cell line that carries an IDH1^{R132C} mutation, but has no effect on the proliferation or viability of fibrosarcoma HT1080 cells in which expression of the IDH1^{R132C} gene is inhibited, such as by expression of a microRNA, siRNA, or antisense RNA or in an HT1080 cell line where the IDH1R132C gene has been disrupted, such as by a knock-out (e.g., deletion) mutation.

The candidate compound can be useful for specifically targeting tumors or treating cancers characterized by IDH mutants that cause overexpression of 2HG. The candidate compounds identified by the selection methods featured in the invention can be further examined for their ability to target a tumor or to treat cancer by, for example, administering the compound to an animal model. For example, an animal model can be a tumor transplant model, e.g., a mouse having an IDH mutation, e.g., IDH1 or IDH2 mutation, mutant cell or tumor transplanted in it. For example, a U87 cell or glioma (e.g., glioblastoma) cell harboring a transfected IDH neoactive mutation, e.g., an IDH1 or IDH2 neoactive mutation, can be implanted as a xenograft and used in an assay. Primary human glioma or AML tumor cells can be grafted into mice to allow propogation of the tumor and used in an assay. A genetically engineered mouse model (GEMM) harboring an IDH1 or IDH2 mutation and/or other mutation, can also be used in further studies.

Detection of 2-hydroxyglutarate

The production of 2-hydroxyglutarate can be monitored by an enzymatic assay, such as a fluorescence-based assay, as described in Example 1 below. The oxidative conversion of NADPH to NADP+ that occurs when alpha-ketoglutarate is reduced to form 2HG can be coupled to a reaction with diaphorase which will provide a means to monitor the reaction by a fluorescet signal. Diaphorase rapidly consumes NADPH to convert resazurin to highly fluorescent resofurin. Thus, a compound that inhibits 2HG production will result in a brighter fluorescent signal.

2-hydroxyglutarate can also be detected by LC/MS or LC-MS/MS (liquid chromotograpy-mass spectrometry/mass spectrometry). To detect secreted 2-hydroxyglutarate in culture media, 500 μL aliquots of conditioned media can be collected, mixed 80:20 with methanol, and centrifuged at 3,000 rpm for 20 minutes at 4 degrees Celsius. The resulting supernatant can be collected and stored at −80 degrees Celsius prior to LC-MS/MS to assess 2-hydroxyglutarate levels. To measure whole-cell associated metabolites, media can be aspirated and cells can be harvested, e.g., at a non-confluent density. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions. Metabolites can be separated by reversed phase chromatography using 10 mM tributyl-amine as an ion pairing agent in the aqueous mobile phase, according to a variant of a previously reported method (Luo et al. J Chromatogr A 1147, 153-64, 2007). One method allows resolution of TCA metabolites: t=0, 50% B; t=5, 95% B; t=7, 95% B; t=8, 0% B, where B refers to an organic mobile phase of 100% methanol. Another method is specific for 2-hydroxyglutarate, running a fast linear gradient from 50% -95% B (buffers as defined above) over 5 minutes. A Synergi Hydro-RP, 100 mm×2 mm, 2.1 μm particle size (Phenomonex) can be used as the column, as described above. Metabolites can be quantified by comparison of peak areas with pure metabolite standards at known concentration. Metabolite flux studies from ¹³C-glutamine can be performed as described, e.g., in Munger et al. Nat Biotechnol 26, 1179-86, 2008.

In an embodiment 2HG, e.g., R-2HG, is evaluated and the analyte on which the determination is based is 2HG, e.g., R-2HG. In an embodiment the analyte on which the determination is based is a derivative of 2HG, e.g., R-2HG, formed in process of performing the analytic method. By way of example such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis. Exemplary 2HG derivatives include dehydrated derivatives such as the compounds provided below or a salt adduct thereof:

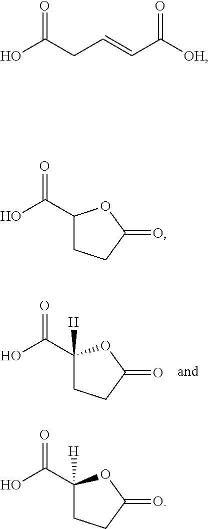 [SMILES: O=C(O)/C=C/CC(=O)O ; O=C1CCC(C(=O)O)O1 ; O=C1CC[C@H](C(=O)O)O1 ; O=C1CC[C@@H](C(=O)O)O1]

Methods of Treatment using the Identified Compounds

The compounds identified by the selection methods featured in the invention can be useful for treating a proliferative disorder in a human, such as a cancer.

As used herein, a “proliferative disorder” is a disorder characterized by unwanted cell proliferation or by a predisposition to lead to unwanted cell proliferation (sometimes referred to as a precancerous disorder). Examples of proliferative disorders include cancers, e.g., cancers characterized by solid tumors, e.g., of the brain, thyroid, colon, prostate, and bone. Exemplary cancers characterized by solid tumors include glioma, follicular thyroid cancer, colon cancer, prostate cancer, fibrosarcoma, osteosarcoma, melanoma, lung cancer (e.g., paraganglioma). Other types of cancers include hematological cancers, e.g., a myeloma, such as multiple myeloma, or a leukemia, such as an acute lymphoblastic leukemia, or an acute myeloid leukemia (also called acute myelogenous leukemia), such as acute monocytic leukemia. Examples of cancers characterized by a predisposition to lead to unwanted cell proliferation include myelodysplasia or myelodysplastic syndrome, which are a diverse collection of hematological conditions marked by ineffective production (or dysplasia) of myeloid blood cells and risk of transformation to AML.

A compound identified by the selection methods can be administered to a human who has a proliferative disorder, such as a cancer, in an amount sufficient to treat the cancer. As used herein, an “effective amount” is the amount of a pharmaceutical composition required to treat a patient suffering from or susceptible to a disease, such as a cancer. The effective amount of a pharmaceutical composition containing the identified compound varies depending upon the manner of administration and the age, body weight, and general health of the subject. Ultimately, the attending prescriber will decide the appropriate amount and dosage regimen. Typically, an effective amount to treat a cancer will cause an improvement in the patient's symptoms, which can be, for example, a decrease in tumor size or a decrease in the amount of cancer cells in the patient, or a decrease in the rate of tumor growth or a decrease in the rate of cancer cell proliferation. An effective amount of the composition containing a candidate compound can also be effective to extend the life of the patient, or to decrease symptoms of the cancer, e.g., fatigue or pain.

A patient administered a compound identified through the selection methods can be evaluated for an effect of the compound on the patient's cancer. The evaluation, which can be performed before and/or after treatment has begun, is based, at least in part, on analysis of a tumor sample, cancer cell sample, or precancerous cell sample, from the subject. The patient can be evaluated for an improvement in cancer symptoms following administration of the compound. An improvement in cancer symptoms can be manifested by, for example, a decrease in tumor size or a decrease in the amount of cancer cells, or a decrease in the rate of tumor growth or a decrease in the rate of cancer cell proliferation or viability. A candidate compound identified by the methods described herein can also be useful to extend the life of the patient, or to decrease symptoms of the cancer, e.g., fatigue or pain.

A candidate compound can also be evaluated in a patient by examining a sample from the patient for the presence or level of a neoactivity product, e.g., 2HG, e.g., R-2HG, by evaluating a parameter correlated to the presence or level of a neoactivity product, e.g., 2HG, e.g., R-2HG. A neoactivity product, e.g., 2HG, e.g., R-2HG, in the sample can be determined by a chromatographic method, e.g., by LC-MS analysis. It can also be determined by contact with a specific binding agent, e.g., an antibody, which binds the neoactivity product, e.g., 2HG, e.g., R-2HG, and allows detection. In an embodiment the sample is analyzed for the level of neoactivity, e.g., a neoactivity, e.g., a 2HG neoactivity. If the compound is found to decrease 2HG levels in the sample, then the compound can be determined to be of benefit to the patient for treatment of the cancer.

In one embodiment the evaluation, which can be performed before and/or after treatment has begun, is based, at least in part, on analysis of a tissue (e.g., a tissue other than a tumor sample), or bodily fluid, or bodily product. Exemplary tissues include lymph node, skin, hair follicles and nails. Exemplary bodily fluids include blood, plasma, urine, lymph, tears, sweat, saliva, semen, and cerebrospinal fluid. Exemplary bodily products include exhaled breath. For example, the tissue, fluid or product can be analyzed for the presence or level of a neoactivity product, e.g., 2HG, e.g., R-2HG, by evaluating a parameter correlated to the presence or level of a neoactivity product, e.g., 2HG, e.g., R-2HG. A neoactivity product, e.g., 2HG, e.g., R-2HG, in the sample can be determined by a chromatographic method, e.g., by LC-MS analysis. It can also be determined by contact with a specific binding agent, e.g., an antibody, which binds the neoactivity product, e.g., 2HG, e.g., R-2HG, and allows detection. In embodiments where sufficient levels are present, the tissue, fluid or product can be analyzed for the level of neoactivity, e.g., a neoactivity, e.g., the 2HG neoactivity.

In one embodiment the evaluation, which can be performed before and/or after treatment has begun, is based, at least in part, on a neoactivity product, e.g., 2HG, e.g., R-2HG, imaging of the subject. In embodiments magnetic resonance methods are is used to evaluate the presence, distribution, or level of a neoactivity product, e.g., 2HG, e.g., R-2HG, in the subject. In one embodiment the subject is subjected to imaging and/or spectroscopic analysis, e.g., magnetic resonance-based analysis, e.g., MRI and/or MRS e.g.,analysis, and optionally an image corresponding to the presence, distribution, or level of a neoactivity product, e.g., 2HG, e.g., R-2HG, or of the tumor, is formed. Optionally the image or a value related to the image is stored in a tangible medium and/or transmitted to a second site. In an embodiment the evaluation can include one or more of performing imaging analysis, requesting imaging analysis, requesting results from imaging analysis, or receiving the results from imaging analysis.

Pharmaceutical Compositions

The compounds identified by the selection methods featured in the invention can be formulated as pharmaceutical compositions for administration to a subject for treatment of a proliferative disorder, such as a cancer characterized by an IDH mutation that causes increased 2HG expression. The compositions delineated herein include the identified compounds, as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions containing the identified compounds may be administered directly to the central nervous system, such as into the cerebrospinal fluid or into the brain. Delivery can be, for example, in a bolus or by continuous pump infusion. In certain embodiments, delivery is by intrathecal delivery or by intraventricular injection directly into the brain. A catheter and, optionally, a pump can be used for delivery. The inhibitors can be delivered in and released from an implantable device, e.g., a device that is implanted in association with surgical removal of tumor tissue. For example, for delivery to the brain, the delivery can be analogous to that with Gliadel, a biopolymer wafer designed to deliver carmustine directly into the surgical cavity created when a brain tumor is resected. The Gliadel wafer slowly dissolves and delivers carmustine.

The pharmaceutical compositions may be administered orally, parenterally, by inhalation, topically, rectally, nasally, buccally, vaginally or via an implanted reservoir, preferably by oral administration or administration by injection. The compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions is useful when the desired treatment involves areas or organs readily accessible by topical application, such as for treatment of a melanoma. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions include a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.02 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

### Example 1

### A Method for Identifying Compounds that have Specific Activity against Mutant IDH1

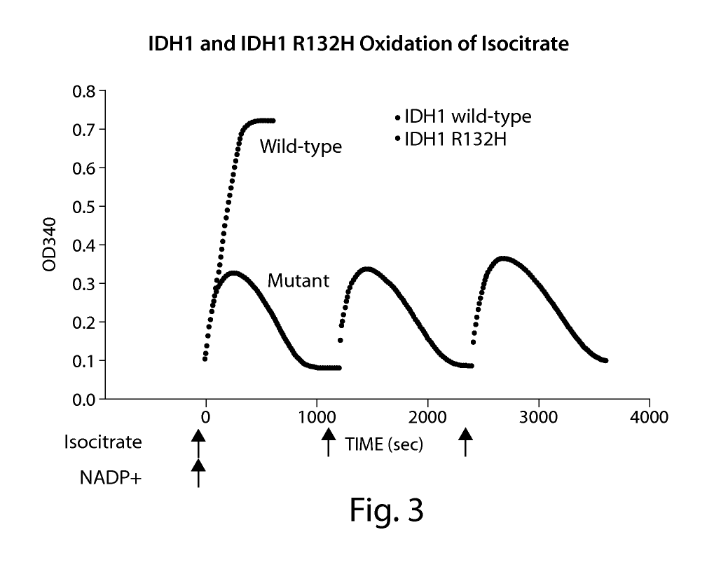

This example describes methods of identifying compounds that have specific activity against mutant IDH1. Detailed enzymology and x-ray structural studies demonstrated that IDH1^{R132H} mutant enzyme gains the neoactivity of reductive conversion of αKG to 2HG utilizing NADPH as cofactor. Kinetic analysis of mutant and wildtype IDH1 activity is illustrated in FIG. 3.

The primary screening method measures the oxidative conversion of NADPH to NADP+ and the consequent reduction of αKG to 2HG. The conversion of NADPH to NADP+ can be assayed by coupling the reaction to diaphorase. This assay can be configured as an end-point readout where excess diaphorase rapidly consumes remaining NADPH and converts resazurin to the highly fluorescent resorufin to generate an increasing signal upon inhibition (λₑₓ=540 nm; λ_{cm}=590 nm). This screening configuration provides specificity in the primary screen. Non-specific compounds, such as reactive or non-specific aggregate inhibitors, are expected to inactivate the diaphorase detection enzyme, preventing the development of a positive signal from the remaining NADPH cofactor. Therefore, this method is expected to minimize the number of false-positives.

Two additional features are incorporated into this assay to further minimize false positives: (i) the inclusion of bovine serum albumin (BSA), and (ii) the use of 2-mercaptoethanol (2-ME) in the reconfirmation step. The inclusion of BSA helps to improve compound solubility and stability, as well as to prevent non-specific aggregate inhibitors. 2-ME is omitted from the primary screen to prevent false positives created through redox cycling of compounds in the presence of reducing agents, but is included in the confirmation step to filter the false positives that may arise through thiol-reactive compounds.

The assay readout is easily detected through the use of a fluorescent plate reader, which detects the fluorescence from the resorufin (λₑₓ=540 nm; λₑₘ=590 nm). Screening plates can be pre-read after the addition of compounds and before the addition of enzyme and substrates where wells containing intrinsically fluorescent compounds may be flagged.

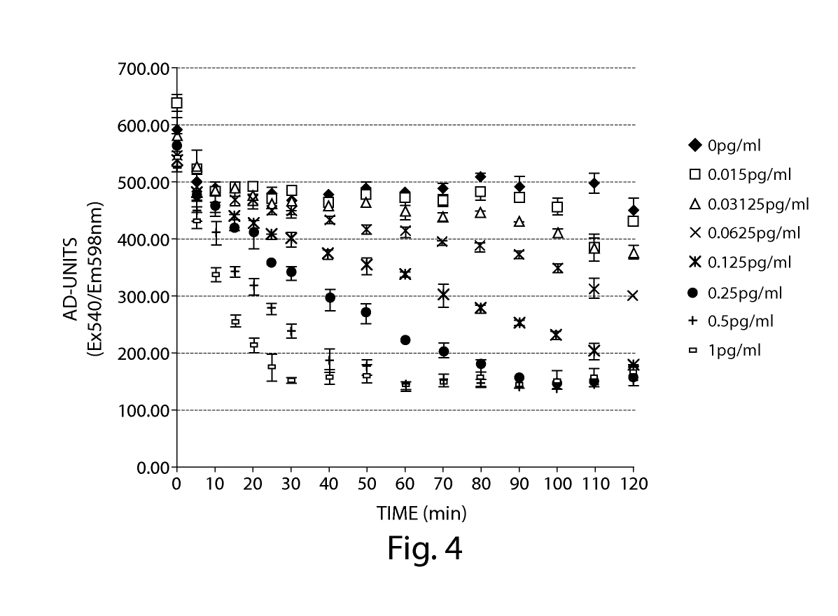

In a preliminary assay, a time-course versus enzyme titration experiment was performed, and the results are shown in FIG. 4. Reactions are performed in 75 μL in a 384-well plate at 25° C. as follows. In 384-well black, opaque bottom, low protein-binding plates, 25 μL enzyme (IDH1R132H) diluted in buffer A (150 mM NaCl, 20 Tris-HCl, pH 7.5, 10 mM MgCl₂, 0.05% (w/v) BSA) is added to 1 μL DMSO or 1 μL of test compound dissolved in DMSO. Then, 25 μL of 2× substrate mixture is added (2 mM αKG, 16 μM NADPH in buffer A) to initiate the reaction. The reaction is stopped and developed by the addition of 25 μL of 12 μM rezasurin and 20 μg/mL diaphorase in buffer A. After 5 mM at 25° C., resorufin fluorescence was measured in a SpectraMax 5e plate reader. Final enzyme concentration of 0.25 μg/mL and reaction time of 90 min were selected.

To ensure that enzyme preparation is well-behaved under DMSO and is stable over the course of the experiments, DMSO tolerance and freeze-thaw stability were tested against the IDH1^{R132H} assay. The enzyme activity was stable for at least six freeze-thaw cycles, and could tolerate up to 5% DMSO without significant loss in signal.

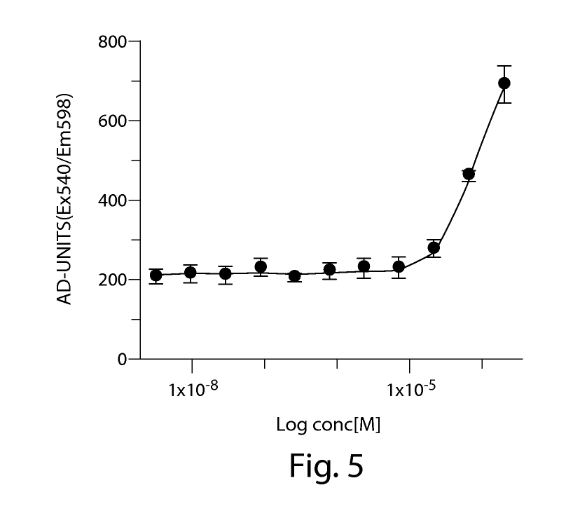

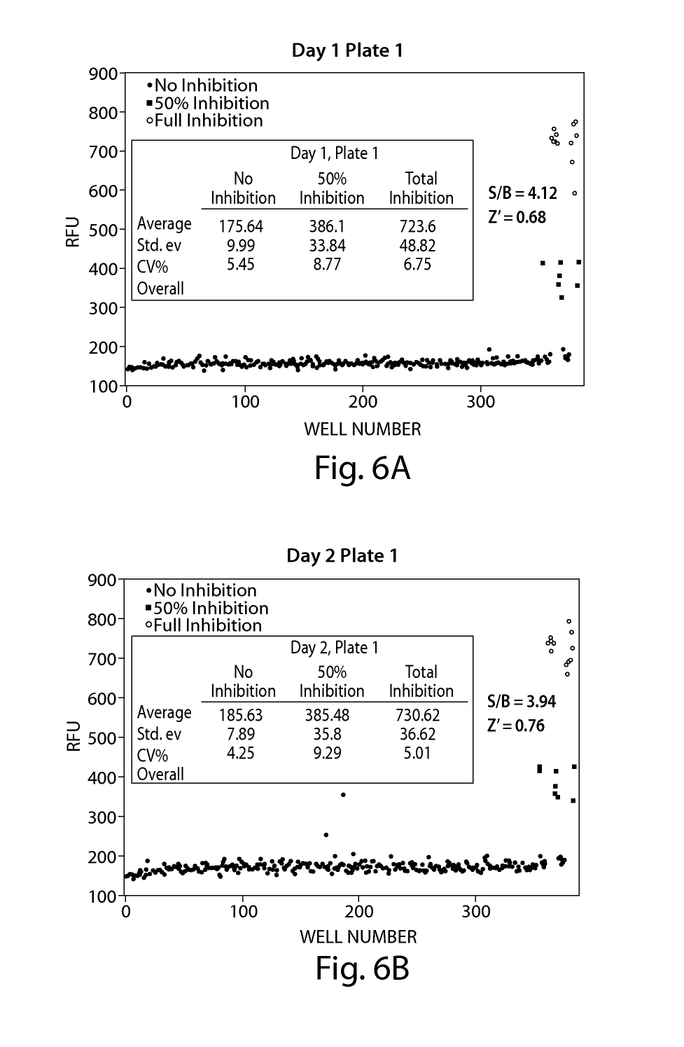

To validate the high through-put screening (HTS) assay, plates were prepared such that wells contained either no inhibitor (to determine total signal), a concentration of suramin (a non-specific dehydogenase inhibitor) (FIG. 5) corresponding to its 1×IC50 values for the 50% INH mark, or a concentration of suramin corresponding to its 4× IC50 values for the 50% INH mark, which would be expected to inhibit the enzyme (IDH1R132H) to near completion, as ˜100% INH mark. Two plates were prepared on Day 1 and one plate on Day 2. The data demonstrated a robust assay with good statistics, well suitable for an HTS campaign (see FIGS. 6A and 6B). In all cases, Z′ was >0.7 and S/B was >4.0. The concordance for the partial inhibition of the reaction was also robust, with day-to-day variation of less than 2%.

One of the strengths of the assay is that a signal for a positive hit requires the diaphorase enzyme to be functional. Inhibitors of diaphorase or non-specific inhibitors (e.g., covalent modifiers, aggregators, reactive ion generators) will also inhibit signal development. Therefore, the false positive rate for this assay is expected to be low. While a discreet selectivity assay to counterscreen against diaphorase is not believed to be necessary, the validation of positive hits include counterscreening against wild-type IDH, and an optional orthogonal UV-based assay.

Active compounds (having specific activity against IDH1R132H) can be selected based upon statistical analysis of results from the primary screen. Actives can be cherry-picked and subject to single-point reconfirmation in duplicate or triplicate in the same primary assay described above, but in the presence of 2 mM 2-ME to eliminate thiol-reactive compounds. Confirmed Actives can be assessed for obvious undesireable chemical moieties using computational methods. The reduced list of compounds can be analyzed with dose titration to generate IC50s, consisting of 11-point 3-fold serial dilutions from 100 μM to 1.7 nM Inhibition data from this titration series can be fit to a four-parameter regression curve, and results examined for quality of the fitness. Compounds with Hill slopes greater than 2 or less than 0.5, or with maximum inhibitions less than 80% may be deprioritized for further studies such as evaluation for mode-of -action against the enzyme (competitive vs substrate/cofactor, reversibility, on-off rates).

In addition, positive hits can be counterscreened against wild-type IDH1 to discover inhibitors against the neomorphic IDH1 mutant activity (e.g., with with >10× selectivity). Such confirmed hits with good selectivity over wild-type IDH1 can be further evaluated for mode-of-action against the enzyme (competitive vs substrate/cofactor, reversibility, on-off rates).

To confirm that the inhibitory activity is directed against IDH1^{R132H} enzyme, an optional UV-based assay can be used to exploit the direct detection of NADPH (λₑₓ340 nm; λₑₘ400 nm) in a decreasing-signal assay as an orthogonal method during the validation phase.

Cell-based assays can be used to further develop and validate the positive hits. Two separate cell-based assays can be used: a mechanism-based assay and a phenotype-based assay. In the mechanism-based assay, a U87 stable cell line is engineered to express a constitutive IDH1^{R132H} mutant protein, which can produce and secrete high quantities of 2HG into the media over time (>16 hrs). The 2HG can be readily quantified by LC-MS/MS. The second assay is phenotype-based and is based on neurospheres of glioma cells derived from gliobastoma patients whose tumors harbor the IDH1^{R132H} mutation and produce high levels of 2HG. The viability of the neurospheres can be measured over a period of >72-96 hrs.

The compounds identified by the methods described herein can be useful as IDH mutant inhibitors, or can be optimized further to be potent and specific inhibitors of IDH1 mutants. Such compounds can therefore be useful for treating patients that harbor IDH mutations (e.g., glioma patients that harbor IDH mutations).

### Example 2

### Methods for Identifying Compounds that Selectively Interfere with Proliferation or Viability of IDH Mutant Cells with Elevated 2HG as Compared to Control Cells that do not have Elevated 2HG

A synthetic-lethal HTS screen is performed using an engineered derivative of the U87MG glioblastoma cell-line that stably and constitutively expresses IDH1^{R132H} mutant enzyme (U87MG-^{R132H} cells). The expression level of IDH1^{R132H} mutant enzyme is comparable to endogenous IDH1 assessed by Western Blots, and produces and secretes high levels of 2HG into media over time (>16 h), which can be readily qualified by LC-MS/MS (FIG. 3). The screen is performed in parallel with parental U87MG cell line that express IDH wildtype and does not produce 2HG as measured by LC-MS/MS method. The activity of candidate compounds on cell is assessed by standard Cell-Titer Glow Assay. Compounds that selectively reduce proliferation or viability of U87MG-R132H cells as compared to the parental U87MG line would be scored as a hit in the primary screen.

A synthetic lethal assay for use in a HTS is designed by adding 30 μl of cell suspension with normal medium (DMEM+10% FBS), at ˜3000/well in a 384-well plate configuration, and the plate placed under normoxia to culture overnight. 1 μL of 400× compound in DMSO can then be added to 99 μL of media, and the mixture mixed gently. Next 10 μL of medium-containing compounds can be added to the cell plate and incubated for 72 hours. To detect a signal, the plates are allowed to equilibrate to room temperature for 30 min before adding 40 μL CTG reagent to each well. The plates are shaken for 2 min, incubated for 10 min at room temperature, and then luminescence is read, such as on a Flexstation3 reader.

The hits from the primary synthetic lethal screen above are validated in paired HT1080 cell lines as described below. HT1080 is a fibrosarcoma cell line that harbors the IDH1R132C mutation (COSMIC database), and constitutively produces high levels of 2HG. A derivative of the HT1080 cells expressing shRNAmir targeting IDH1 under the control of the doxycyclin-inducible TRE promoter was generated. Treatment of doxycyclin (DOX) in the IDH1-knockdown line significantly reduces the production of 2HG. Hits identified in the primary screen can be evaluated in untreated or doxocyclin treated HT1080 cells harboring the IDH1 shRNAmir and a non-silencing shRNAmir for additional control.

To further characterize the activity of hit compounds, the neurosphere lines HK217 and HK252 (established in the Kornblum lab at UCLA), carrying wildtype and mutant IDH1 respectively, can be used as a second confirmatory screen, insuring that mutant-selective growth inhibition is observed in these independent backgrounds, thus assessing the behaviour of these compounds across diverse cellular backgrounds.

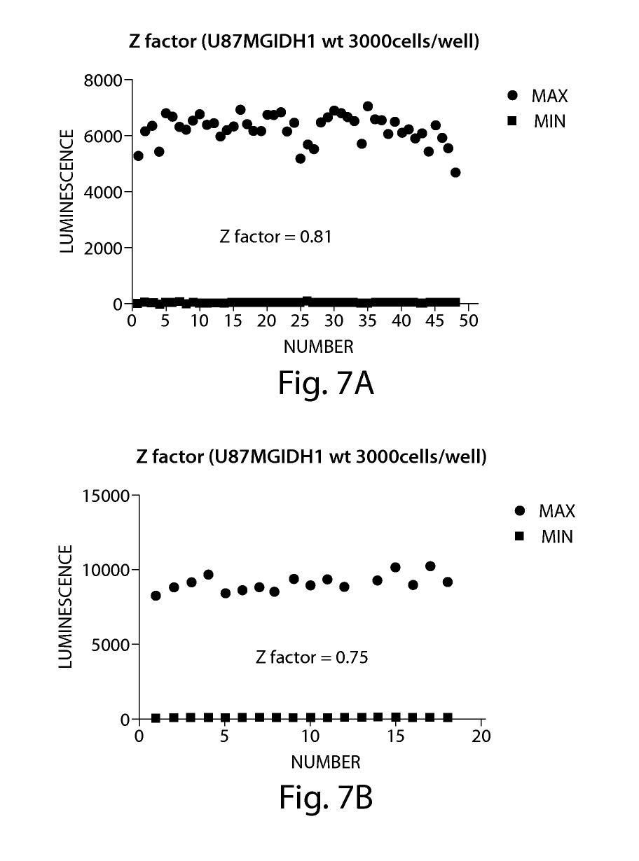

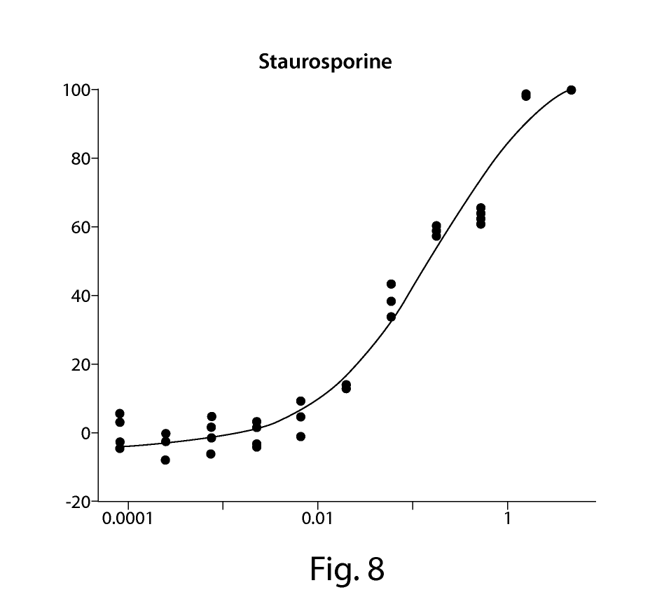

In preliminary assays, U87MG^{R132H} or parental U87MG cell lines were first tested for signal quality and reproducibility. Two plates at either 2000 or 3000 cells/well density were tested in parallel with either DMSO or staurosporine, a generic cytotoxic agent (see also FIG. 8). After 72 hours of incubation with the compounds in a culture incubator, cells were evaluated for viability using a standard Cell-Titer Glow kit. Either 2000 or 3000 cells/well density gave good intraplate reproducibility with Z factor >0.7 for either parental U87MG or U87MG^{R132H} lines. However, higher density gave higher signal leading to better overall S/B ratio, and therefore 3000 cells were chosen for incubation at 72 hrs to further optimize the assay. FIGS. 7A and 7B show the consistency of performance of the U87MG match pair cell lines (parental and R132H) in the 384-well format.

To confirm that the U87MG^{R132H} cells are responsive to generic cytotoxic agents, staurosporine was tested as a positive control. As expected, a titration of staurosporine gave a dose-responsive viability effect with EC50 ˜100 nM (FIG. 8).

All publications, patents, and patent applications mentioned in this specification are herein incorporated by reference to the same extent as if each independent publication or patent application was specifically and individually indicated to be incorporated by reference.

A number of embodiments have been described. Nevertheless, it will be understood that various modifications may be made without departing from the spirit and scope of the invention. Accordingly, other embodiments are within the scope of the following claims.

What is claimed is:

1. A method of identifying a candidate compound that selectively interferes with proliferation or viability of a first IDH (isocitrate dehydrogenase)-mutant cell that has elevated levels of 2HG (2-hydroxyglutarate) comprising contacting a candidate compound with a first IDH-mutant cell that has elevated levels of 2HG, and if proliferation or viability of the first IDH-mutant cell that has elevated levels 2HG is decreased as compared to a control cell that does not have elevated levels of 2HG, then identifying the candidate compound as a compound that interferes with proliferation or viability of the first IDH-mutant cell.

2. The method of claim 1, wherein the first IDH-mutant cell carries a mutation in the IDH1 gene.

3. The method of claim 1, wherein the first IDH-mutant cell carries a mutation in the IDH2 gene.

4. The method of claim 1, wherein the first IDH-mutant cell carries an IDH1^{R132X} mutation.

5. The method of claim 1, wherein the first IDH-mutant cell carries an IDH2^{R172X} mutation.

6. The method of claim 1, wherein the first IDH-mutant cell carries an IDH1^{R132H} mutation.

7. The method of claim 1, wherein the first IDH-mutant cell carries an IDH2^{R172K} mutation.

8. The method of claim 1, wherein the first IDH-mutant cell carries an IDH1^{R132C} mutation.

9. The method of claim 1, wherein the first IDH-mutant cell carries an IDH2 ^{R140Q} mutation.

10. The method of claim 1, wherein the first IDH-mutant cell carries any one of the mutations listed in Table 1.

11. The method of claim 1, wherein the first IDH-mutant cell is from a cancer cell line.

12. The method of claim 1, wherein the first IDH-mutant cell is from a glioma cell line or a fibrosarcoma cell line.

13. The method of claim 1, wherein the first IDH-mutant cell is from a U87 glioma cell line.

14. The method of claim 1, wherein the first IDH-mutant cell is from a U87 glioma cell line engineered to express IDH1^{R132H}.

15. The method of claim 1, wherein the first IDH-mutant cell is from a fibrosarcoma /HT1080 cell line that carries an IDH1^{R132C} mutant gene.

16. The method of claim 14, wherein the control cell is a parental U87 cell.

17. The method of claim 15, wherein the control cell is a fibrosarcoma HT1080 cell engineered to expressed a microRNA, siRNA, or antisense RNA that inhibits expression of the IDH1^{R132C} mutant gene.

18. The method of claim 1, further comprising testing the candidate compound for tumor suppressor activity in an animal model.

19. The method of claim 1, further comprising administering the compound to a human who has a cancer, in an amount sufficient to treat the cancer.

20. The method of claim 1, wherein the cell proliferation is assayed by luminescence.

21. The method of claim 1, wherein the candidate compound is obtained from a library.

22. The method of claim 1, further comprising testing the selected candidate compound for an ability to selectively interfere with proliferation or viability of a second IDH-mutant cell comprising contacting the selected candidate compound with a second IDH-mutant cell that has elevated levels of 2HG, and if proliferation or viability of the second IDH-mutant cell is decreased as compared to a second control cell that does not have elevated levels of 2HG, then identifying the candidate compound again as a compound that selectively interferes with proliferation or viability of the first IDH-mutant cell.

23. The method of claim 22, wherein the first IDH-mutant cell is from a U87 cell line that carries an IDH1^{R132H} mutation, and the second IDH-mutant cell line is an HT1080 cell line.

24. A method of identifying a compound that specifically interferes with an IDH (isocitrate dehydrogenase)-mutant enzyme that causes elevated levels of 2HG (2-hydroxyglutarate) comprising contacting a candidate compound with an IDH-mutant cell that has elevated levels of 2HG, and if 2HG levels are decreased as compared to a control cell that does not have elevated levels of 2HG, then identifying the candidate compound as a compound that specifically interferes with the IDH-mutant enzyme.

25. The method of claim 24, wherein 2-HG production is assayed by an enzymatic fluorescence assay.

26. The method of claim 24, further comprising testing the selected candidate compound for an ability to selectively interfere with proliferation or viability of the IDH-mutant cell, comprising contacting the selected candidate compound with the IDH-mutant cell, and if proliferation or viability of the IDH-mutant cell is decreased as compared to the control cell, then identifying the candidate compound as a compound that selectively interferes with proliferation of the IDH-mutant cell.
